(19) United States
(12) Patent Application Publication
(10) Pub. No.: US 2005/0085692 A1
(43) Pub. Date: Apr. 21, 2005

(54) Endoscope

(76) Inventors: Ralf Kiehn, Seitingen-Oberflacht (DE); Oliver Heiko Rehe, Tuttlingen (DE); Thomas Paul Weller, Tuttlingen (DE); Martin Knight, Lancashire (GB)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/967,063

(22) Filed: Oct. 15, 2004

(30) Foreign Application Priority Data
Oct. 17, 2003 (EP) 03 023 645.9

(51) Int. Cl. A61B 1/012

(52) U.S. Cl. 600/130; 600/128; 600/138; 600/153

(57) ABSTRACT

An endoscope comprising a shaft having distal and proximal ends is provided, which shaft comprises an outer tube in which an inner tube is inserted such that it is rotatable relative to the outer tube and displaceable in the longitudinal direction of the tubes and such that a first channel is formed between the two tubes, with an instrument tube and imaging optics being arranged in the inner tube and a second channel being formed therein, and with both the instrument tube and the imaging optics being connected with the inner tube in a manner locked against rotation relative to each other.

### BACKGROUND OF THE INVENTION

1. Field of the Invention

The invention relates to an endoscope used, in particular, in the medical field.

2. Background

When operating on the vertebral column, for example, endoscopes have to be as small as possible and well sterilizable.

Therefore, it is an object of the invention to provide an endoscope which comprises a shaft being as small as possible in diameter and having several functions at the same time.

According to the invention, this object is achieved by an endoscope comprising a shaft having distal and proximal ends, which shaft comprises an outer tube in which an inner tube is inserted such that it is rotatable relative to the outer tube and displaceable in the longitudinal direction of the tubes and such that a first channel is formed between the two tubes. Said endoscope further comprises a main part connected with the proximal end of the shaft, with an instrument tube as well as imaging optics being arranged in the inner tube and a second channel being formed therein. Both the instrument tube and the imaging optics are connected with the inner tube so as to be locked against rotation relative to each other.

Since the instrument tube and the imaging optics are connected with the inner tube in a manner locked against rotation relative to each other, the instrument tube and the imaging optics can be rotated relative to the outer tube, which is advantageous, in particular, for use of the endoscope in the medical field. However, for rotatability of the instrument tube and of the imaging optics, it is not required, in the endoscope according to the invention, that the imaging optics and the instrument tube be rotatably arranged relative to the inner tube. This advantageously enables a relatively free choice of cross-sectional shapes and sizes for the instrument tube and the imaging optics.

Thus, using the endoscope according to the invention, the region at the distal end can be viewed by means of the imaging optics. At the same time, manipulation is possible within this region by means of an instrument arranged in the instrument tube. Further, the two channels can also be used as rinsing and suction channels. Thus, different functions are realized in the endoscope according to the invention.

In the endoscope according to the invention, both the instrument tube and the imaging optics are preferably connected with the inner tube such that they are not displaceable relative to the inner tube in a longitudinal direction.

The instrument tube, the imaging optics and the inner tube thus form a unit which is rotatable and longitudinally displaceable relative to the outer tube.

By providing two separate channels in the shaft of the endoscope, one of said channels may be used as a rinsing channel, through which a rinsing liquid can be supplied to the region located at the distal end of the shaft, and the other channel may be used as a suction channel, through which the rinsing liquid is, in turn, sucked off at the distal end of the shaft.

In particular, the outer tube may be open at the distal end and may have a chamfered shape relative to the longitudinal direction of the shaft, as viewed in a lateral view. This also makes the distal end itself usable as an instrument, which is useful, in particular, for operations on the vertebral column as an instrument for pushing aside nerves or for protection of nerves against an instrument being employed, through the instrument tube, at the distal end in the region to be operated on.

In particular, both the instrument tube and the imaging optics are releasably connected with the inner tube. This facilitates sterilization and, in particular, autoclaving of the endoscope. In this regard, the endoscope may be further embodied such that the outer tube can also be removed completely from the inner tube, so that the outer tube and the inner tube can also be sterilized separately.

Further, in the endoscope according to the invention, a first shutoff valve can be arranged at the proximal end of the shaft or of the outer tube, respectively, said first shutoff valve communicating with the first channel. This shutoff valve is preferably arranged to be rotatable about the longitudinal direction or about an axis parallel to the longitudinal direction, respectively. This allows the shutoff valve to be brought into a desired position relative to the shaft.

Further, a second shutoff valve can be arranged at the proximal end of the shaft or of the inner tube, respectively, said second shutoff valve communicating with the second channel. The second shutoff valve is preferably also arranged to be rotatable about the longitudinal direction or about an axis parallel to the longitudinal direction, respectively. Thus, the second shutoff valve can also be positioned relatively freely relative to the shaft.

The combination of the shutoff valves with the feature that the instrument tube and the imaging optics are releasably connected with the inner tube has the advantage that the instrument tube and the imaging optics can be exchanged without having to remove and re-attach flexible tubes connected with the shutoff valves. This leads to improved ergonomics, in particular in medical applications.

Particularly preferably, the inner tube has an oval cross-section. On the one hand, this ensures rotatability of the inner tube relative to the outer tube and, on the other hand, the first channel is provided between the outer tube and the inner tube.

Further, in the endoscope according to the invention, the inner tube may be open at the distal end and may be chanfered relative to the longitudinal direction, as viewed in a lateral view.

It is particularly preferred, in the endoscope according to the invention, if the instrument tube and the imaging optics are arranged within an endoscope tube which is, in turn, inserted in the inner tube such that the second channel is formed between the inner tube and the endoscope tube. This design realizes the entire assembly of tubes in a very space-saving manner.

It is then particularly advantageous, if the endoscope tube has an oval cross-section selected such that the maximum clearance in the region of the instrument tube is greater than the maximum clearance in the region of the imaging optics, as viewed in cross-section. This allows an instrument tube to be provided with a larger cross-sectional area than the imaging optics, so that the space for the instruments increases.

In a preferred embodiment of the endoscope according to the invention, a sealing system is provided at the proximal end of the instrument tube, said sealing system sealing the proximal end of the instrument tube, whether an instrument is introduced or not.

For this purpose, the sealing system may comprise two sealing units, with the first sealing unit providing sealing when an instrument is inserted and the second sealing unit providing sealing when no instrument is inserted. The two sealing units are preferably arranged behind each other, as viewed in a longitudinal direction of the instrument tube.

In particular, the endoscope according to the invention may be further embodied such that the instrument tube and the imaging optics are connected with each other and form a first endoscope unit, and that a second endoscope unit comprising a further instrument tube and further imaging optics is provided, said endoscope units being insertable in the inner tube in an alternating manner and connectable with the inner tube in the inserted condition. Thus, an endoscope or an endoscope system, respectively, is provided wherein the endoscope units are easily exchangeable. Since the inserted endoscope units are exchangeably provided, such exchange may be effected, for example, during the intended use of the endoscope (for example, during an operation).

The second endoscope unit may be further embodied in the same manner as the above-described first endoscope unit. Both endoscope units may differ, in particular, in one feature. This feature may be, for example, the viewing direction of the imaging optics with respect to the longitudinal direction.

Of course, the endoscope system may comprise more than two endoscope units, which preferably differ in at least one feature (e.g. the viewing direction of the imaging optics).

To provide a connection between the respective endoscope unit and the inner tube, a locking unit may be provided at the main part (preferably at the proximal end of the main part) by which the desired connection can be realized and also released again.

### BRIEF DESCRIPTION OF THE DRAWINGS

The invention will be explained in more detail below, by way of example and with reference to the Figures, wherein:

FIG. 1 shows a first embodiment of the endoscope according to the invention in a first operative position,

FIG. 2 shows the endoscope of FIG. 1 in a second operative position,

FIG. 3 shows the section A-A of the shaft of FIG. 1,

FIG. 4 shows a view of the endoscope unit of the endoscope of FIGS. 1 and 2, and

FIG. 5 shows an enlarged sectional view of the sealing system at the proximal end of the endoscope of FIGS. 1 and 2,

FIG. 6 shows a representation of a cross-recessed seal of the sealing system of FIG. 5, and

FIG. 7 shows an enlarged sectional view of the quick-locking unit of the endoscope shown in FIGS. 1 and 2.

### DETAILED DESCRIPTION

The endoscope comprises a shaft 1 having a distal end 2 and a proximal end 3 as well as a main part 4.

The shaft 1 comprises an outer tube 5 having a length of about 16 cm with a circular cross-section having an external diameter of about 7.5 mm into which an inner tube 6 with an oval cross-section is inserted. The cross-section of the inner tube 6 is selected such that, in the inserted condition, as is best seen in FIG. 3, a first channel 7, in this case a suction channel, is formed between the inner and outer tubes 6, 5. At the proximal end 8 of the outer tube 5, a grip portion 9 connected with the outer tube 5 is provided, said grip portion 9 carrying a shutoff valve 10 comprising a connector 11 (e.g a Luer lock). Fluid communication between the connector 11 and the first channel 7 can be established or interrupted by means of the shutoff valve 10. The shutoff valve 10 (together with the connector 11) is connected with the grip portion 9 such that it is rotatable about the longitudinal axis L of the shaft 1.

A sleeve 12, into which the proximal end of the inner tube 6 is inserted, is attached to the main part 4. Attachment of the inner tube 6 within the sleeve 12 can be effected by means of soldering or welding. Alternatively, it is also possible to form the main part 4 and the sleeve 12 in one single piece. On the outside of the sleeve 12, the grip portion 9 is supported so as to be displaceable in a longitudinal direction L and rotatable such that the outer tube 5 is displaceable and rotatable relative to the inner tube 6. The sleeve 12 comprises an annular seal 13 which, in addition to the desired sealing of the proximal end of the first channel 7, simultaneously also serves to determine the necessary force required to displace and/or rotate the outer tube 5 relative to the inner tube 6 in a longitudinal direction of the shaft 1.

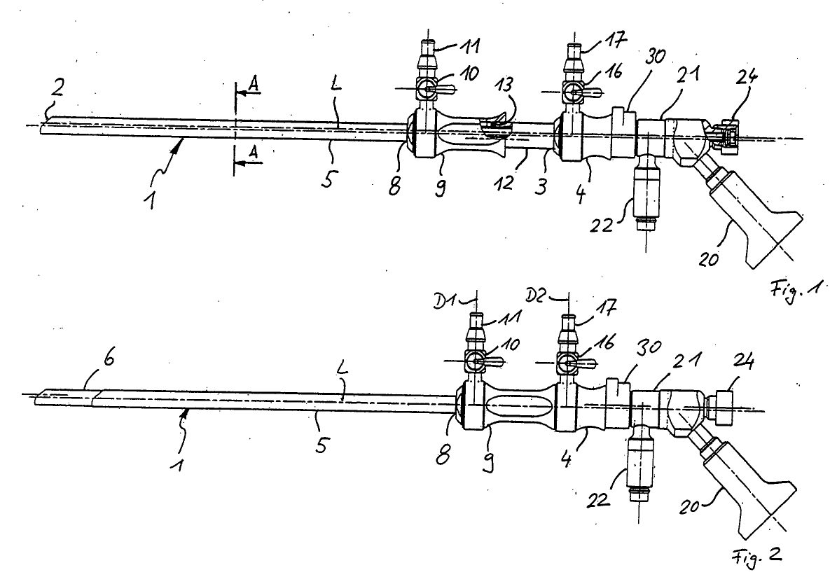

FIG. 1 shows the endoscope in a first end position, wherein the distal ends of the inner and outer tubes 6, 5 are on the same level. FIG. 2 shows a second end position, wherein the distal end of the inner tube 6 protrudes out of the outer tube 5.

The use of the sleeve 12 makes it possible to provide a thin-walled inner tube 6 having a small cross-sectional area for the shaft 1, which is advantageous, in particular, in medical applications (for example, operations on the vertebral column). The required stability for guiding the grip portion 9 in the longitudinal displacement and/or in the rotation of the outer tube 5 relative to the inner tube 6 is then provided by the sleeve 12, which has a greater wall thickness.

As is best seen in FIG. 3, an endoscope tube 14 having an oval cross-section is inserted in the inner tube 6. The cross-section of the endoscope tube 14 is selected such that a second channel 15 is formed between the inner tube 6 and the endoscope tube 14. At the main part 4, a shutoff valve 16 comprising a connector 17 is provided for the second channel 15. Fluid communication between the second channel 15 and the connector 17 can be established or interrupted by means of the shutoff valve 16. In the presently described embodiment example, the second channel 15 serves as a rinsing channel. The shutoff valve 16 and the connector 17 are arranged at the main part 4 so as to be rotatable about the longitudinal axis L of the shaft 1.

An instrument tube 18 having a circular cross-section and an optical tube 19 having a circular cross-section are arranged inside the endoscope tube 14, with the diameter of the optical tube 19 being smaller than that of the instrument tube (here, 2.3 mm versus 3.7 mm). The oval shape of the endoscope tube 14 is selected such that the maximum clearance W1 in the region of the instrument tube 18 corresponds substantially to the external diameter of the optical tube 19. Thus, as is evident from FIG. 3, the endoscope tube 14 has a substantially egg-shaped cross-section.

Optical fibers (not shown), which serve to illuminate the object to be recorded, are also arranged inside the endoscope tube 14 and between the instrument tube 18 and the optical tube 19. In the optical tube 19, lens optics are arranged, which are known from optical endoscopes and which transmit the recorded image to an eyepiece 20 arranged at the proximal end of the endoscope. Of course, instead of the lens optics, an electronic image sensor preceded, if necessary, by an optical system, may be provided at the distal end of the optical tube 19, for example, said sensor then transmitting the image signals via an electronic line to the eyepiece 20, which may then comprise a corresponding image display unit.

The optical tube 19, the instrument tube 18 and the endoscope tube 14 are securely connected with an end piece 21 (for example, by soldering or welding) at the proximal end of the endoscope tube 14. A connector 22, through which light can be directed into the optical fibers, is attached to the end piece 21. Further, the eyepiece 20, through which either the recorded image can be viewed directly, or to which a camera (not shown) can be connected, is releasably connected with the end piece 21. This has the advantage that, if a camera comprising a sterile cover is attached to the eyepiece 20, as may happen during operations, the eyepiece 20 can be removed (together with the camera) from the endoscope tube when exchanging the endoscope tube 14 and can then be immediately attached again to the new endoscope tube.

The instrument tube 18 extends through the end piece 21 and is sealed with a sealing system 24 such that the proximal end of the instrument tube 18 is sealed, whether an instrument is inserted or not. For this purpose, as is best seen in FIG. 5, the sealing system 24 comprises a rubber sealing cap 25 with a central hole 26 whose size is selected such that, with an instrument inserted therein, sealing is effected by means of the sealing cap 25, and the sealing system 24 also comprises, for example, two cross-recessed silicone seals 27 and 28 (FIG. 6) which, in their mounted condition, are rotated relative to each other such that they have a sealing effect in case no instrument is inserted.

The endoscope tube 14, the instrument tube 18, the optical tube 19, the optical fibers and the end piece 21, which comprises the connector 22, the eyepiece 20 and the sealing system 24, form an endoscope unit 29. The described endoscope unit 29 is a so-called 30° unit, because the recording direction B, as schematically shown in FIG. 4, is inclined 30° relative to the longitudinal direction. The shaft 1 of the endoscope comprises the outer tube 5, the inner tube 6, the sleeve 12 and the corresponding parts of the endoscope unit 29 inserted therein.

For example, in order to allow a quick exchange of the endoscope unit 29 during an operation (without having to remove the entire endoscope from the region to be operated on), because the surgeon needs a 0° endoscope unit (viewing direction along the longitudinal direction), for example, the endoscope unit 29 is connectable with the inner tube 6 by means of a quick-locking unit 30 arranged at the main part 4, so that the endoscope tube 14 is connected with the inner tube 6 in a manner locked against rotation relative to each other, and a longitudinal displacement between both tubes 14 and 6 is not possible.

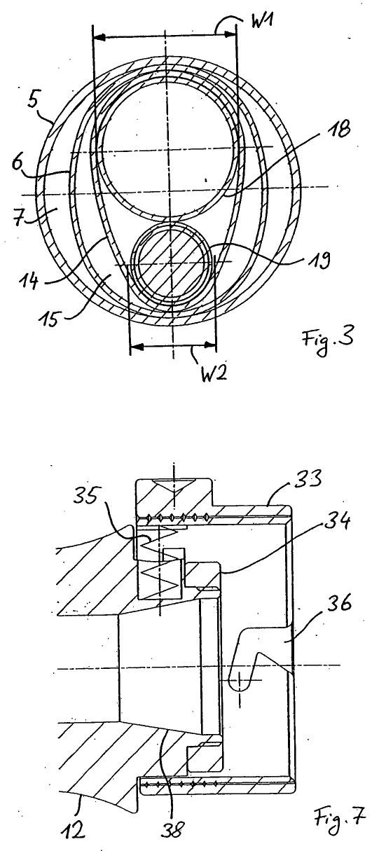

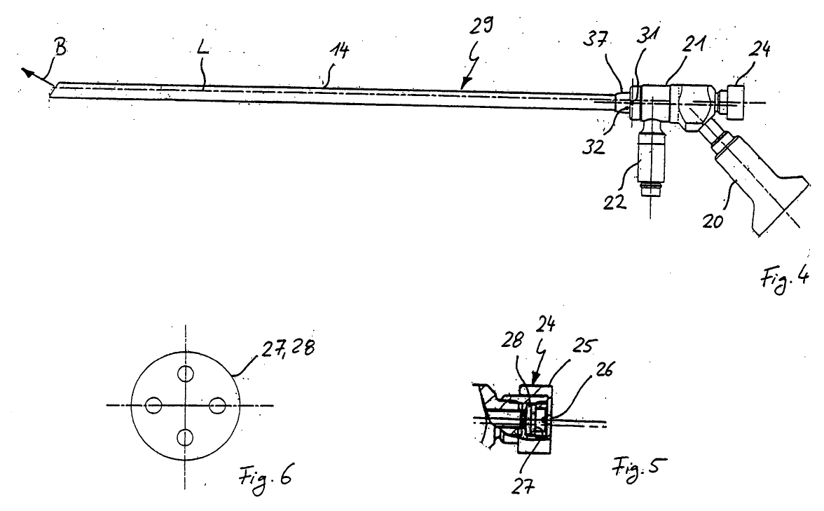

First, description is made of how to insert the endoscope unit 29. The end piece 21 comprises two locking pins 31 (of which only one is visible in FIG. 4) as well as a guiding pin 32, as is best seen in FIG. 4. The quick-locking unit 30, as is best seen in FIG. 7, comprises a grip portion 33 which is attached to the sleeve 12 by a nut 34 such that it is fixed in the axial direction. In the radial direction shown in FIG. 7, the grip portion 33 is displaceable by being moved toward the indicated locking position by means of a spring 35 arranged between the sleeve 12 and the grip portion 33. The grip portion 33, as seen in FIG. 7, can be pushed down against the elastic force and thus moved into the unlocking position. The proximal end of the grip portion 33 is provided with one milled portion or with two milled portions 36, respectively, (of which only one is visible in FIG. 7) for the locking pins 31 as well as a guiding groove (not shown) for the guiding pin 32.

In order to connect the endoscope tube 14 with the inner tube 6, the endoscope tube 14 is inserted into the inner tube 6 from the proximal side, with the guiding pin 31 then engaging the longitudinal groove of the quick-locking unit 30. The shape of the milled portion 36 is selected such that, when the endoscope tube 14 is being pushed in, the locking pin 31 pushes the grip portion 33 down, as viewed in FIG. 7, against the elastic force of the spring 35. If the endoscope tube 14 is pushed in further, the locking pin 31 enters into a locking portion of the milled portion 36, so that, due to the restoring force of the spring 35, the grip portion 33 is pushed upward again and thus effects locking of the endoscope tube 14 relative to the inner tube 6 in the longitudinal direction of the shaft 1. The guiding pin 31 engaging with said groove locks the connection in rotation, so that the endoscope tube 14 is connected with the inner tube 6 in a manner locked against rotation relative to each other and not displaceable in a longitudinal direction. Thus, when moving the inner tube 6 relative to the outer tube 5, the distance between the shutoff valve 16 and the connector 22 and the eyepiece 20 does not change. In the connection thus established, a conical portion 37 of the endoscope unit 29 contacts the conical sealing surface 38 of the sleeve in a sealing manner such that the proximal end of the second channel 15 is sealed.

In order to release the endoscope unit 29, the grip portion 33 merely has to be pushed down against the spring 35, and then the endoscope tube 14 needs to be pulled out of the inner tube 6 toward the right (in FIGS. 1 and 2), by its end piece 21.

In order to effect the described exchange, the grip portion is pushed down and the endoscope unit 29 is pulled out. If there is still an instrument present in the instrument tube 18, it is preferably removed before pulling out the endoscope unit 29. Where a camera is attached to the eyepiece 20, it may also be separated from the eyepiece, or it may be separated from the endoscope unit together with the eyepiece (if required), before pulling out the endoscope unit 29. Subsequently, the 0° endoscope unit is inserted in the described manner and fixed by means of the quick-locking unit 30. Thereupon, the instrument may then be inserted in the instrument channel and the camera (if desired) may be attached to the end piece. Of course, endoscope units having other inclinations of the recording direction, e.g. 45° and 70°, may also be used. A particular advantage of said exchange is that flexible tubes connected with the connectors 11, 17 need not be removed and re-attached, so that said exchange can be quickly effected.

The endoscope comprising the endoscope unit 29 and the further 0° endoscope unit forms an endoscope system wherein an exchange of the endoscope unit is easy to perform during use of the endoscope. This is possible due to the modular structure of the endoscope.

The distal end 2 of the outer tube 5 is chamfered as seen in the side view of FIGS. 1 and 2. Thus, the distal end can also be used directly as an instrument in operations. The distal end of the inner tube 6 is chamfered in the same way, too.

The endoscope is provided such that it can be completely dismantled. Thus, the outer tube 5 can be completely withdrawn from the inner tube 6, together with the grip portion 9. As described herein, the endoscope tube 14 can also be separated from the inner tube 6. This allows the tubes to be separately cleaned and sterilized. This is particularly advantageous in autoclaving.

In order to enable a secure grip on the grip portion, the grip portion comprises four flat sides, as indicated in the representations of FIGS. 1 and 2.

1. An endoscope comprising a shaft having distal and proximal ends, said shaft comprising an outer tube in which an inner tube is inserted such that said inner tube is rotatable relative to the outer tube and displaceable in a longitudinal direction of the outer and inner tubes and such that a first channel is formed between the outer and inner tubes, an instrument tube and imaging optics arranged in the inner tube and a second channel being formed between the inner tube and the instrument tube, both the instrument tube and the imaging optics connected with the inner tube in a manner locked against rotation relative to each other.

2. The endoscope of claim 1, wherein both the instrument tube and the imaging optics are connected with the inner tube such that they are not displaceable relative to the inner tube in the longitudinal direction.

3. The endoscope of claim 1, wherein the outer tube is open at the distal end and is chamfered, as viewed in a lateral view, relative to the longitudinal direction of the shaft.

4. The endoscope of claim 1, wherein both the instrument tube and the imaging optics are releasably connected with the inner tube.

5. The endoscope of claim 1, wherein a first shutoff valve is arranged at the proximal end of the shaft, said first shutoff valve communicating with the first channel.

6. The endoscope of claim 5, wherein said first shutoff valve is arranged so as to be rotatable about the longitudinal direction or about an axis parallel to the longitudinal direction.

7. The endoscope of claim 6, wherein said first shutoff valve is connected with the inner tube such that it is not displaceable relative to the inner tube in the longitudinal direction.

8. The endoscope of claim 5, wherein a second shutoff valve is arranged at the proximal end of the shaft, said second shutoff valve communicating with the second channel.

9. The endoscope of claim 8, wherein said second shutoff valve is arranged so as to be rotatable about the longitudinal direction or about an axis parallel to the longitudinal direction.

10. The endoscope of claim 9, wherein said second shutoff valve is connected with the inner tube such that said second shutoff valve is not displaceable relative to the inner tube in the longitudinal direction.

11. The endoscope of claim 1, wherein the inner tube has an oval cross-section.

12. The endoscope of claim 1, wherein the inner tube is open at the distal end and is chamfered, as viewed in a lateral view, relative to the longitudinal direction.

13. The endoscope of claim 1, wherein the instrument tube and the imaging optics are arranged within an endoscope tube, said endoscope tube inserted in the inner tube such that the second channel is formed between the inner tube and the endoscope tube.

14. The endoscope of claim 13, wherein the endoscope tube has an oval cross-section, with the maximum clearance in the region of the instrument tube being larger than the maximum clearance in the region of the imaging optics, as viewed in cross-section.

15. The endoscope of claim 1, wherein a sealing system is arranged at the proximal end of the instrument tube, said sealing system sealing the proximal end of the instrument tube, whether an instrument is inserted in the instrument tube or not.

16. The endoscope of claim 1, wherein the instrument tube and the imaging optics are connected with each other and form a first endoscope unit, and wherein a second endoscope unit comprising a further instrument tube and further imaging optics is provided, said endoscope units each being insertable in the inner tube and being connectable with the inner tube in the inserted condition.

17. An endoscope with a distal end and a proximal end, the endoscope comprising:
an outer tube;
an inner tube disposed in the outer tube, a first channel defined between the outer tube and the inner tube;
an instrument tube disposed in the inner tube, a second channel defined between the inner tube and the instrument tube;
a seal disposed proximate the endoscope distal end;
means for viewing an object proximate the distal end of the endoscope; and
means for ingressing and egressing a fluid into, and out of, the endoscope.

18. The endoscope of claim 17, wherein said viewing means is a 30 degree unit.

19. The endoscope of claim 17, wherein said viewing means is a 0 degree unit.

20. The endoscope of claim 17, in which said viewing means comprises an end eye piece.

21. The endoscope of claim 17, further comprising a quick locking unit at least partially securing the viewing means to the endoscope.

22. The endoscope of claim 17, in which the outer and inner tubes are chamfered.

23. The endoscope of claim 17, in which the ingressing and egressing means includes a first connector conveying a fluid to one of the first and second channels and a second connector conveying a partial vacuum to the other of the first and second channels.
